(19) United States
(12) Patent Application Publication
(10) Pub. No.: US 2018/0153230 A1
(43) Pub. Date: Jun. 7, 2018

(54) OZONE GARMENT SYSTEM AND METHODS THEREOF

(71) Applicant: Marolina Outdoor Inc., Charleston, SC (US)

(72) Inventors: Benjamin D. Verner, Jarrettsville, MD (US); Devin T. Sweeney, Mount Pleasant, SC (US); John A. Holschuh, Perry Hall, MD (US)

(21) Appl. No.: 15/819,035

(22) Filed: Nov. 21, 2017

Related U.S. Application Data
(60) Provisional application No. 62/429,932, filed on Dec. 5, 2016.
(60) Provisional application No. 62/444,750, filed on Jan. 10, 2017.

(51) Int. Cl. A41D 13/00; A41D 1/02; A41D 27/20; A01M 31/00; A61L 9/12

(52) U.S. Cl. CPC A41D 13/0002; A41D 1/02; A41D 27/20; A61L 2209/134; A61L 9/12; A41D 2400/36; A61L 2209/212; A01M 31/008

(57) ABSTRACT

A scent control system comprises a garment system having a cloaking substance dispersion system including a dispersion circuit for providing cloaking substance for cloaking the user.

### CROSS-REFERENCE TO RELATED APPLICATIONS

The present application claims the benefit of and priority to U.S. Provisional Patent Application Nos. 62/429,932, filed Dec. 5, 2016, and 62/444,750, filed Jan. 10, 2017, the contents of each of which are hereby incorporated by reference in their entireties, including but without limitation, those portions directed to ozone dispersion devices, systems, and methods.

### BACKGROUND

The present disclosure relates generally to devices, systems, and methods of reducing detection, and more specifically to devices, systems, and methods of reducing scent detection.

Controlling scents of persons and/or equipment can be desirable in a variety of settings. Managing scents uncommon to a particular area can decrease the risk of detection. For example, hunters often spend considerable efforts to reduce, control, and/or otherwise manage scents which might indicate to game animals that a threat is nearby. Such scent control can be particularly important when the potential game has sensitive olfactory facilities.

### SUMMARY

The present disclosure may comprise one or more of the following features and combinations thereof.

According to an aspect of the present disclosure, a garment system for dispersion of cloaking substance may include at least one garment for wearing by a user, the at least one garment including a jacket, a dispersion circuit coupled with the jacket, the dispersion circuit defining a dispersion pathway including at least one inlet and at least one outlet, a dispersion guidance system secured with the jacket proximate the at least one outlet to direct flow of cloaking substance away from a user's face. The garment system may include a cloaking substance delivery assembly fluidly connected with the at least one inlet of the dispersion circuit to conduct cloaking substance through the dispersion pathway for discharge through the at least one outlet to cloak the jacket with cloaking substance.

In some embodiments, the at least one garment includes at least one other garment. Each of the jacket and the at least one other garment may have conduit. The conduit of the jacket may be selectively connected with the conduit of the at least one other garment to define the dispersion circuit for passing cloaking substance to cloak the at least one garment.

In some embodiments, the dispersion guidance system may include a flap extending over an exterior of the jacket to define a pocket space. The at least one outlet may be arranged within the pocket space. The dispersion guidance system may include an opening formed between the flap and the exterior of the jacket for guiding cloaking substance out from the outlet. The opening may be directed away from a face opening of the jacket to direct the cloaking substance away from the user's face. In some embodiments, he opening may be directed downward.

In some embodiments, the garment system may include a dispersion control system. The dispersion control system may include a region selection device connected with the dispersion circuit. The region selection device may be operable for selective communication with at least two regions of the garment to selectively provide cloaking substance to the at least two regions.

In some embodiments, the region selection device may be operable in a first position to communicate cloaking substance with the at least two regions. The region selection device may be operable in a second position to block communication of cloaking substance to at least one of the at least one regions. In some embodiments, one of the at least two regions may be an upper region of the jacket. Another of the at least two regions may be a lower region of the jacket.

In some embodiments, one of the at least two regions may be a region of another garment having conduit that is selectively connected as part of the dispersion circuit. The region selection device may be connected with the dispersion circuit to selectively provide cloaking substance to the region of the another garment.

According to another aspect of the present disclosure, a garment ozone dispersion system for providing a cloak of ozone to prevent detection by game may include at least one garment for wearing by a user, a dispersion circuit coupled with the at least one garment, the dispersion circuit defining a dispersion pathway including at least one inlet and at least one outlet. A dispersion guidance system may be secured with the at least one garment. The dispersion guidance system may be arranged proximate the at least one outlet to direct flow of cloaking substance away from a user's face. A cloaking substance delivery assembly fluidly may be connected with the at least one inlet of the dispersion circuit to conduct cloaking substance through the dispersion pathway for discharge through the at least one outlet to cloak the garment with cloaking substance. In embodiments including the dispersion guidance system, the cloaking substance may discharge through the at least one outlet to the dispersion guidance system.

In some embodiments, the at least one garment may include a plurality of garments each having conduit. The conduit of each one of the plurality of garments may be selectively connected with the conduit of the remainder of the plurality of garments to define the dispersion circuit for passing cloaking substance to cloak the plurality of garments.

In some embodiments, the dispersion guidance system may include a flap extending over an exterior of the at least one garment to define a pocket space. The at least one outlet may be arranged within the pocket space.

In some embodiments, the dispersion guidance system may include an opening formed between the flap and the exterior of the at least one garment for guiding cloaking substance out from the outlet. The opening may be directed away from a face opening of the garment to direct the cloaking substance away from the user's face. In some embodiments, the opening may be directed downward.

In some embodiments, the garment ozone dispersion system may include a dispersion control system. The dispersion control system may include a region selection device connected with the dispersion circuit. The region selection device may be operable for selective communication with at least two regions of the garment to selectively provide cloaking substance to the at least two regions.

In some embodiments, the region selection device may be operable in a first position to communicate cloaking substance with the at least two regions. The region selection device may be operable in a second position to block communication of cloaking substance to at least one of the at least one regions. One of the at least two regions may be an upper region of the garment. Another of the at least two regions may be a lower region of the garment.

In some embodiments, one of the at least two regions may be a region of another garment having conduit that is selectively connected as part of the dispersion circuit. The region selection device may be connected with the dispersion circuit to selectively provide cloaking substance to the region of the another garment.

According to another aspect of the present disclosure, a garment system for providing a cloak of ozone to prevent detection by game may include at least one garment for wearing by a user, a dispersion circuit coupled with the at least one garment, the dispersion circuit defining a dispersion pathway including at least one inlet and at least one outlet, a cloaking control system including a controller coupled with the at least one garment. The controller may be adapted to determine preferred parameters of ozone dispersion to cloak the at least one garment.

In some embodiments, the garment system may include a dispersion control system adapted to govern flow of ozone through the dispersion circuit. The dispersion control system may be arranged in communication with the controller. The controller may adapted to determine a preferred flowrate of ozone for emission through the at least one outlet.

In some embodiments, the controller may be in communication with a flow control device of the dispersion control system. The flow control device may be coupled with the dispersion circuit to control a flowrate of ozone to the at least one outlet. The controller may be adapted to communicate at least one signal to the flow control device to execute a preferred position to achieve the preferred flowrate of ozone.

In some embodiments, the cloaking control system may include circuitry for communicating with a personal mobile device. The cloaking control system may be operable to receive user inputs from the personal mobile device. The controller may determine the preferred flowrate of ozone based on the user inputs.

These and other features of the present disclosure will become more apparent from the following description of the illustrative embodiments.

### BRIEF DESCRIPTION OF THE DRAWINGS

FIG. 1 is a perspective front view of a garment (jacket) having an ozone dispersion system including a dispersion circuit formed of a network of conduit arranged about the jacket, and a cloaking substance delivery system housed in a pocket of the jacket and connected with the dispersion circuit for distributing a cloaking substance, such as ozone, through the conduit network to cloak the jacket with the cloaking substance, and showing that the jacket includes a dispersion guidance system including a material flap for guiding dispersion of the cloaking substance;

FIG. 2 is a perspective view of a portion of the dispersion guidance system of the jacket of FIG. 1 including the material flap that extends over a portion of the exterior of the jacket to define a inner space to receive conduit of the dispersion circuit, the flap having an open side (lower side) to direct cloaking substance away from the user's face, and showing that conduit is installed into the inner space;

FIG. 3A is a perspective view of a flow control element of the dispersion circuit of FIG. 1 for governing flow of the cloaking substance through the ozone dispersion system;

FIG. 3B is a front view of a region selector of the dispersion circuit of FIG. 1 for regulating the areas (or zones) of the jacket to which the cloaking substance flows through dispersion circuit and showing that the region selector includes three positions corresponding to three outlets for selecting a low region only, high region, and another region on other garments having other conduit connected to selectively receive cloaking substance;

FIG. 4 is a perspective rear view of another pocket of the jacket of FIG. 1 having a power source, shown as a battery, arranged therein and coupled with a cable to provide electric power to the delivery system;

FIG. 5 is a perspective rear view of the garment of FIG. 1 showing another pocket of the jacket in which a battery is housed to provide electric power to the delivery system;

FIG. 6 is a perspective view of the delivery system of FIG. 1 and in partial explosion from the pocket to show that the delivery system includes a housing (rendered partially transparent) including a cavity therein with an inlet and outlet and showing that the delivery system includes a pressure source for drawing external air into the cavity as feed air to pressurize the housing, a generator module for forming ozone as the cloaking substance from a portion of the feed air for dispersion through the dispersion circuit;

FIG. 7 is a perspective view of the delivery system of FIG. 6 showing curved exterior surfaces of the housing;

FIG. 8 is a perspective view of the delivery system of FIG. 7 with the housing rendered partially transparent to show that the pressure source and the generator module, and power circuitry are arranged within the cavity, and showing that power circuitry is electrically connected with the generator module to provide suitable power to generate ozone;

FIG. 9 is a top perspective view of the delivery system of FIGS. 5, 7, and 8 showing that the housing has curvature to form a flask shape and showing that the outlet includes a connector for connection with the dispersion circuit;

FIG. 10 is a bottom perspective view of the delivery system of FIGS. 5 and 7-9 showing that the inlet includes a tapered opening and a screen to prevent ingress of foreign substances;

FIG. 11 is a bottom perspective view of another embodiment of a delivery system adapted for use in the ozone dispersion system of FIG. 1 similar to that of FIGS. 5 and 7-9 but having a smaller inlet suitable for use with a pump;

FIG. 12 is a top perspective of the delivery system of FIG. 11;

FIG. 13 is a front perspective view of the delivery system of FIGS. 11 and 12 showing the housing partially transparent to show that the pressure source is formed as the pump having an intake extending for connection with the inlet;

FIG. 14 includes various views of the garment of FIGS. 1-6 showing the jacket having camouflage pattern to reduce the risk of sight detection;

FIG. 15 is a perspective view of a covering of another embodiment of a delivery system as shown in FIGS. 16 and 17 in isolation to show that it includes openings for access to control features and an air openings to pass feed air;

FIG. 16 is perspective view of another embodiment of a cloaking substance delivery system adapted for use in the ozone dispersion system of FIG. 1 showing the housing with the covering removed to show that the housing includes a dispersion control system having a flow control device and a region selector arranged in-situ with the housing;

FIG. 17 is a perspective view of the delivery system of FIG. 16 having the cover installed over the housing and showing that the flow control device and the region selector are accessible through the access openings;

FIG. 18 is a perspective view of the delivery system of FIGS. 16 and 17 showing that the housing can be inserted into a complimentary pocket of the jacket and showing that the pocket includes control openings for access to the dispersion control system while the housing is within the pocket;

FIG. 19 is a perspective view of the delivery system of FIGS. 16-18 showing that the pocket includes an inner flap having an operation opening for permitting access to the dispersion control system while securing the housing within the pocket and an outer flap arranged in an open position for accessing the dispersion control system;

FIG. 20 is a perspective of the delivery system of FIGS. 16-19 showing that the outer flap is secured in a closed position to cover the dispersion control system;

FIG. 21 includes various views of the delivery system of FIGS. 18-20 showing the pocket of the jacket having a camouflage pattern,

FIG. 22 is a front view of another embodiment of the garment (jacket) having conduit of the dispersion circuit secured with the jacket in a vine style for dispersing cloaking substance from the cloaking substance delivery system, the conduit including a free end adapted for selectively connection with conduit of other garments as a portion of the dispersion circuit to provide cloaking substance thereto to cloak the jacket;

FIG. 23 is a rear view of the jacket of FIG. 22 further showing the network of conduit of the dispersion circuit;

FIG. 24 is a front view of another garment (pants) including conduit attached to the pants for selective connection with the conduit on the jackets of FIGS. 1 and 22 to fluidly connect as a part of the dispersion circuit of a scent control system;

FIG. 25 is a rear view of the pants of FIG. 24;

FIG. 26 is a perspective view of another garment (boot) including conduit for connection with the conduit on the pants of FIGS. 24 and 25 to fluidly connect as a part of the dispersion circuit of the scent control system;

FIG. 27 is a perspective view of a hunting tool embodied as a hunting blind (shelter) in which a hunter would hide, a dispersion circuit having conduit coupled with the hunting blind and including a passageway for passing cloaking substance therethrough, and an cloaking substance delivery system having a source for providing cloaking substance through the dispersion circuit to cloak the blind with cloaking substance;

FIG. 28 is a perspective view of another garment (mask) and conduit attached to the mask for selective connection with the conduit on the jacket of FIGS. 1 and 22 to fluidly connect as a part of the dispersion circuit;

FIG. 29 is a perspective view of another garment (beanie) and conduit attached to the beanie for selective connection with the conduit on the jacket of FIGS. 1 and 22 to fluidly connect as a part of the dispersion circuit;

FIG. 30 is a perspective view of another garment (namely a cap) and conduit attached to the cap for selective connection with the conduit on the jacket of FIGS. 1 and 22 to fluidly connect as a part of the dispersion circuit;

FIG. 31 is a perspective view of another garment (backpack) and conduit attached to the backpack for selective connection with the conduit on the jacket of FIGS. 1 and 22 to fluidly connect as a part of the dispersion circuit

FIG. 32 is a front view of another garment (vest) which can be substituted or used in conjunction with the jacket of FIGS. 1 and 22 to selectively connect with other garments and provide cloaking substance thereto;

FIG. 33 is a rear view of the vest of FIG. 32;

FIG. 34 is a diagrammatic view of a communications system of the jacket of FIGS. 1 and 22 showing that the jacket includes a communications controller in communication with dispersion control system to govern the flow of cloaking substance, and showing that the controller is in communication with various sensors for determining flow governance, and showing that the controller is in communication with a personal mobile device to receive user input for flow governance.

### DETAILED DESCRIPTION OF THE DRAWINGS

For the purposes of promoting an understanding of the principles of the disclosure, reference will now be made to a number of illustrative embodiments illustrated in the drawings and specific language will be used to describe the same.

Scent management can be an important aspect of decreasing the risk of detection. Managing scents to decrease the risk of detection, for example, for hunters, can be a time consuming and/or laborious task requiring many steps and much care. Even careful control of portable scents, for example, scents emanating from clothing and/or equipment, can fail to avoid detection by game animals. Moreover, scents generated by the hunter, for example, odor from sweat and/or breath may persist.

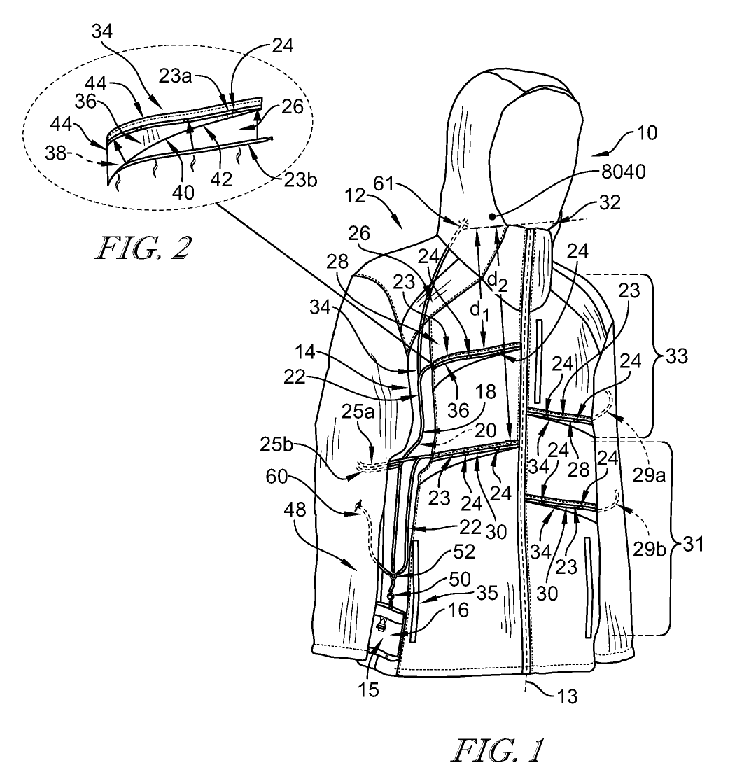

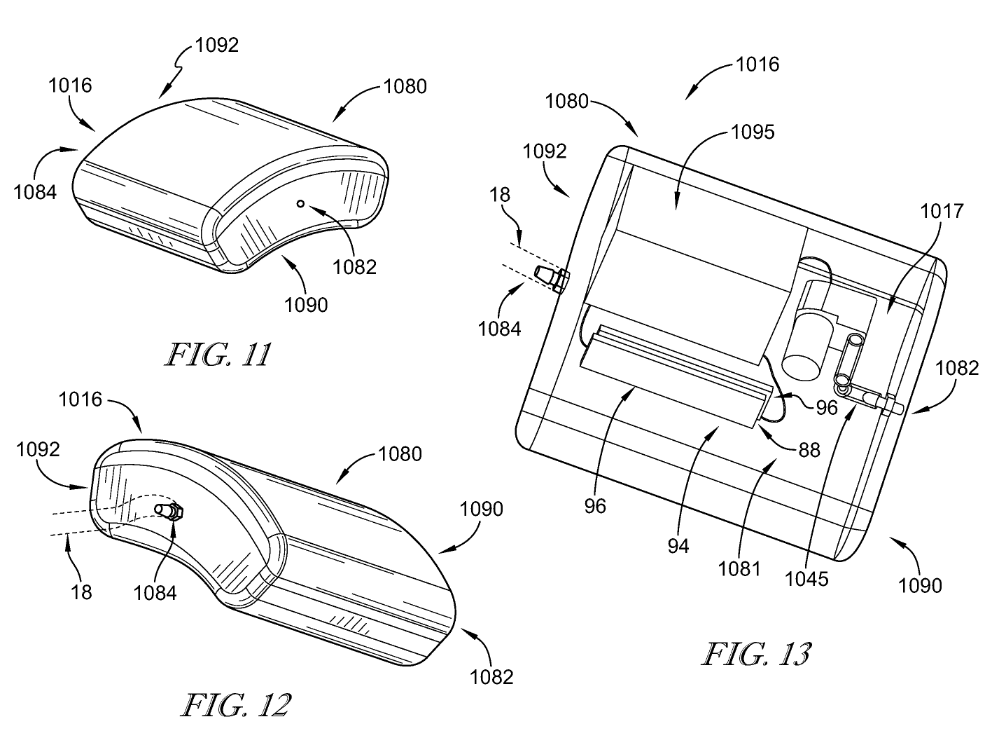

In the illustrative embodiments as shown in FIGS. 1-34, a scent management system includes a cloaking substance dispersion system for providing a cloak of substance to reduce risk of scent detection. The cloaking substance dispersion system is illustratively embodied as an ozone dispersion system 10 providing ozone for cloaking.

As shown in FIG. 1, the dispersion system 10 illustratively includes a garment, embodied as a jacket 12, a dispersion circuit 14 coupled to the jacket 12, and a cloaking substance delivery system 16 for providing cloaking substance through the dispersion circuit 14. The features of the dispersion system may be applied in a garment system comprising one or more articles of clothing for selective interconnection to disperse cloaking substance, as discussed in additional detail herein.

As mentioned above, the cloaking substance is illustratively embodied to be ozone providing an ozone cloak about the garment and the user inhibiting the effects of scents that could indicate the user's presence. The delivery system 16 (housed within pocket 15) illustratively includes a pressure source for providing ozone gas for cloaking as described in additional detail below. In some embodiments, the cloaking substance may include any of ozone and/or other agents, materials, carriers, and/or combinations thereof for reducing detection and/or attracting game.

As shown in FIG. 1, the dispersion circuit 14 illustratively includes a network of tubing or conduit 18 defining a flow passageway 20 therein for distributing ozone for cloaking. The conduit 18 illustratively includes interconnected sections 22, 23 spanning across the jacket 12 to form the network. The dispersion circuit 14 is fluidly connected with the delivery system 16 to receive ozone for dispersion and cloaking.

In the illustrative embodiment, the conduit 18 includes outlets 24 formed as perforations distributed along the conduit 18 that are fluidly connected with the passageway 20 through which ozone can be expelled to cloak the jacket 12. In the illustratively embodiment, the sections 23 of the conduit 18 include the outlets 24 illustratively shown as distinct openings throughout the figures, but in some embodiments, the sections 23 may include breathable material that allows ozone to emanate along their lengths as an effective plurality of outlets 24. In some embodiments, the outlets 24 may have any suitable arrangement, size, and/or configuration to emit ozone. In some embodiments, the sections 22 of the conduit 18 may not include outlets 24 and/or may extend internal to the jacket 12, for example, internal to an exterior 26 of the jacket 12.

Referring to FIG. 1, sections 23 of the conduit 18 each illustratively extend from connection with sections 22. In the illustrative embodiment, the sections 23 of the conduit 18 include an upper section 28 and a lower section 30. The upper section 28 and the lower section 30 each illustratively extend along the front of the jacket 12 from connection with a respective section 22 to a terminal point near a centerline 13 of the jacket 12 (illustratively including a zipper). In the illustrative embodiment, on the right hand portion of the front of the jacket 12 from the user's perspective, the upper section 28 is generally arranged at a distance d₁ from a face opening 32 of the jacket 12 and the lower section 30 is generally arranged at a distance d₂ from the face opening 32, d₁ being smaller than d₂ such that the upper section 28 is positioned closer to the user's face, while d₂ is positioned away from the user's face. On the left hand portion of the front of the jacket 12 from the user's perspective, the upper section 28 is generally arranged at a distance d₃ from the face opening 32 of the jacket 12 and the lower section 30 is generally arranged at a distance d₄ from the face opening 32, d₃ being smaller than d₄ such that the upper section 28 is positioned closer to the user's face, while d₄ is positioned away from the user's face. The distance d₃ is illustratively larger than the distance d₁ but smaller than the distance d₂, and the distance d₄ is larger than the distance d2. Accordingly, the upper and lower sections 28,30 of the right and left portions of the front of the jacket 12 are alternating in distance from the face opening 32 creating an interleaved arrangement along the front of the jacket 12 for cloaking.

Referring now to FIGS. 1 and 2, the jacket 12 illustratively includes a dispersion guidance system 34 for assisting dispersion of ozone. The dispersion guidance system 34 illustratively includes a flap 36 extending over a portion of the exterior 26 of the jacket 12 to define a space 38 for receiving a respective section 23 of the conduit 18 therein (as indicated by the installed position 23a of the section 23 compared to the uninstalled position 23b shown in FIG. 2). The flap 36 can protect the section 23 from any of sight, contact (e.g., contact with body parts, equipment, plants, and the like) and/or exposure to environmental elements (e.g., exposure to fluids, precipitation, wind, dust, dirt, and the like).

In the illustrative embodiment, the flap 36 includes an edge 40 that remains unsealed from the exterior 26 of the jacket 12 to define an opening 42 therebetween. The opening 42 fluidly connects the space 38 with atmosphere to disperse ozone from the outlets 24 to the atmosphere to cloak the jacket 12. The flap 36 is illustratively formed in close conformance with the exterior 26 establishing the space 38 as an envelope slot and having a sleek outer contour conforming with the contour of the jacket exterior 26. The opening 42 is illustratively embodied as a slim slot, that is expandable according to relative movement of the exterior 26 and the flap 36, permitting communication between the space 38 and atmosphere while the flap 36 maintains a low profile outer contour.

In the illustrative embodiment as shown in FIGS. 1 and 2, the openings 42 are generally directed away from the face opening 32 of the jacket 12 to encourage ozone away from the user's face. In the illustrative embodiment, the opening 42 is directed generally downward from connection with the space 38, but in some embodiments, may be directed in any suitable direction. In the illustrative embodiment, other edges 44 of the flap 36 may be secured to the exterior 26 of the jacket 12 to at least partially or fully seal and encourage ozone to exit the space 38 through the opening 42. The section 23 is illustratively secured into the installed position 23a by stitching, but in some embodiments, may be releasably secured into the installed position 23a by ties, fasteners, snaps, any other suitable manner, and/or combinations thereof.

Referring to the illustrative embodiment of FIG. 2, the flap 36 is formed to have a wedge shape in the lateral direction across the jacket 12. The edge 40 illustratively includes slightly concave curvature which tapers the height of the flap 36 proceeding proximally toward the centerline 13 of the jacket 12. The section 23 is illustratively disposed beneath the flap 36 (in the installed position 23a) in an upper area of the space 38, in the orientation of FIG. 2, away from the edge 40 to permit ozone to accumulate in the space 38 before emission through the opening 42. In the illustrative embodiment, each of the outlets 24 of the section 23 are orientated towards the opening 42 (e.g., downward in the orientation of FIG. 2), but in some embodiments, one or more of the outlets 24 may be orientated at least partially towards the exterior 26 of the jacket 12, outwards from the exterior 26 towards the interior of the flap 36, towards the centerline 13 of the jacket 12, away from the centerline 13 of the jacket 12 and/or with any other suitable orientation.

The flap 36 itself is illustratively formed of the same or similar material as the exterior 26 of the jacket 12, and may be water repellant and/or water proof. The flap 36 is illustratively formed to block ozone from passing therethrough, but in some embodiments, the flap 36 may itself be at least partially perforated and/or breathable to permit some amount of cloaking substance to dispel through the flap 36 itself. For example, the flap 36 may include a breathable material disposed along the edge 40 to permit ozone to pass therethrough while other portions of the flap 36 prevent passage of ozone therethrough. In such embodiments, the flap 36 may be secured to the exterior 26 of the jacket 12 at the edge 40 to enclose the space 38 while permitting ozone to pass into the atmosphere thorough the breathable material disposed along the edge 40.

As shown in FIG. 1, the delivery system 16 is illustratively housed within an interior of the jacket 12, namely within a pocket 15. The pocket 15 is illustratively lined with a sound dampening material, such as plush fabric and/or foam, to suppress noise from the delivery system 16. Conduit 18 illustratively extends into the pocket 15 for connection with the delivery system 16.

In the illustrative embodiment as shown in FIG. 1, the conduit 18 is generally hidden from view with the interior of the jacket 12 but is shown through the jacket 12 for descriptive purposes. For example, the sections 22 are disposed inside of the exterior 26 of the jacket 12 and the sections 23 extend from connection with the respective sections 22 within the jacket 12 to outside the exterior 26 within the respective space 38 of the flap 36. As described above, the section 23 is covered by the flap 36 in the installed position 23a. A dispersion control system 48 that is connected as a portion of the dispersion circuit 14, as described below, is illustratively secured with the jacket 12 and is accessible on the exterior 26 of the jacket 12 for user operation.

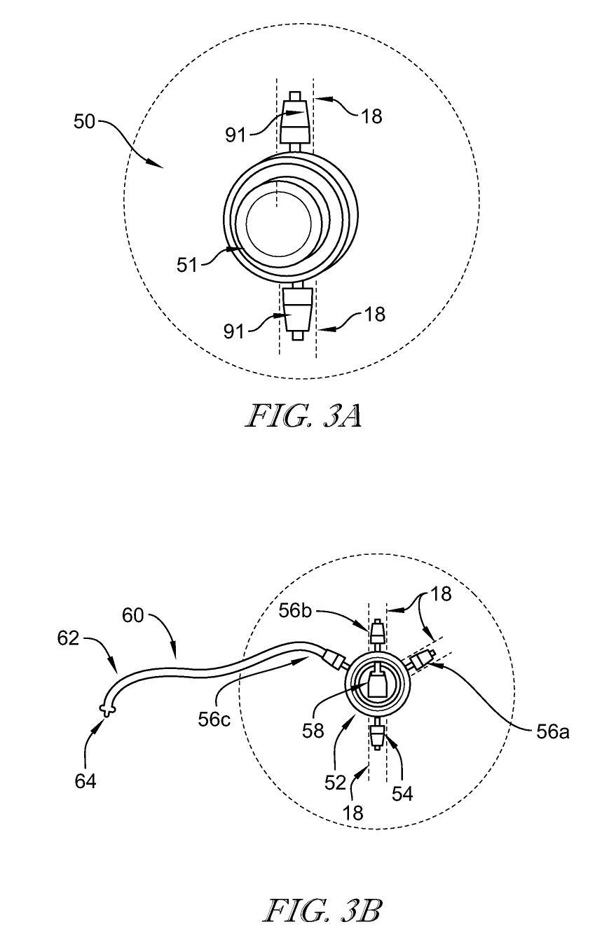

As shown in FIGS. 1 and 3A, the dispersion circuit 14 includes the dispersion control system 48 for governing the flow of ozone through the network of conduit 18. The dispersion control system 48 illustratively includes a flow control device 50 connected with conduit 18 by male connectors 91 in series between the delivery system 16 and the outlets 24. The flow control device 50 is illustratively embodied as an adjustable flow device, namely a ball valve, but in some embodiments may include any of a ball valve, a gate valve, a butterfly valve, a pinch/squeeze valve, a needle valve, other valves and/or dampers, and/or combinations thereof. In some embodiments, the flow control device 50 may include any suitable type and/or arrangement to control flow and/or pressure of ozone through the passageway 20. In the illustrative embodiment, the flow control device 50 is manually operable into various positions between a fully opened (100% open) position to permit regulated flow of ozone through the passageway 20 and a fully closed (0% open) position to block flow of ozone, but in some embodiments, may be operable through a more limited range of positions. The flow control device 50 is embodied as manually adjustable by rotating dial 51 through its range of positions, but in some embodiments, the flow control device 50 may be electrically actuated and/or automatically controlled to achieve its desired position, for example, a flow rate of ozone relative to wind speed and/or a user determined parameter.

Referring to FIG. 3B, the dispersion control system 48 illustratively includes a region selector 52 for designating which portions of the conduit 18 receive a flow of ozone. In the illustrative embodiment, the region selector 52 is an adjustable flow splitter having an inlet 54 connected to receive ozone from the delivery system 16 and a number of outlets 56a, 56b, 56c each connected by a male connectors 91 with certain sectors of the network of conduit 18 that are selectively placed in communication with the inlet 54 according to adjustment of the region selector 52. The region selector 52 is illustratively embodied as a rotary spool valve, but in some embodiments, may include any suitable adjustable flow splitter device. In the illustrative embodiment, the region selector 52 includes a selection switch 58 that is selectively operable between three distinct positions to designate the flow of ozone. In some embodiments, a shut-off position may be included to block flow of ozone to all regions. In some embodiments, the region selector 52 may include any suitable number of outlets 56 and the selection switch 58 may include any suitable number of distinct operational positions to selectively provide ozone to various regions of the jacket 12 and/or to various connected garments as discussed herein.

In a first position of the region selector 52, the switch 58 (when aligned with the first outlet 56a) fluidly connects the inlet 54 only with the first outlet 56a. The first outlet 56a is illustratively connected to sections 22, 23 corresponding to a low region 31 of the jacket 12 including the lower sections 30 of conduit 18. Placing the region selector 52 in the first position illustratively connects the outlets 24 of the lower sections 30 with the delivery system 16 to disperse ozone only from the lower sections 30 (including both of the sections 23 disposed farther from the face opening 32 on the right and left chest regions from the user's perspective, as shown in FIG. 1) and thus generally in the lower region 31 of the jacket 12.

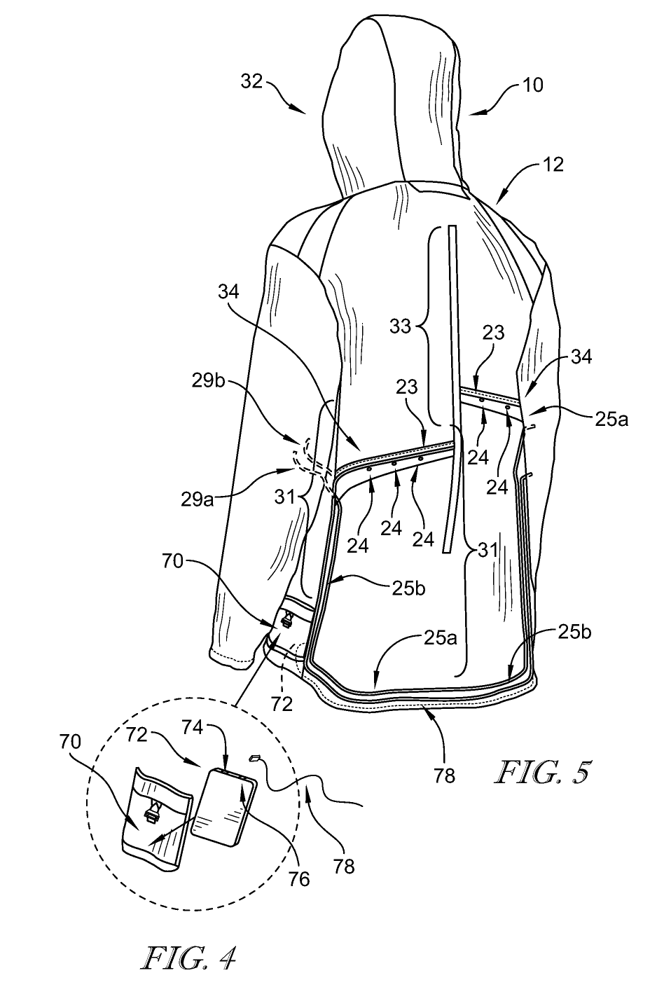

In a second position of the region selector 52, the switch 58 is illustratively aligned with the second outlet 56b as shown in FIG. 4B. In the second position, the region selector 52 illustratively connects the inlet 54 with each of the first and second outlets 56a and 56b. The second outlet 56b is illustratively connected to the sections 22, 23 corresponding to an upper region 33 comprising the upper section 28 of conduit 18 (and including both of the sections 23 disposed closer to the face opening 32 on the right and left chest regions from the user's perspective, as shown in FIG. 1). Placing the region selector 52 in the second position illustratively connects the upper and lower regions 31, 33 with the delivery system 16 to disperse ozone from each of the upper and lower regions 31, 33 (only) and, thus, generally in the upper and lower regions 33, 31 of the jacket 12.

In a third position of the region selector 52, the switch 58 is illustratively aligned with the third outlet 56c. In the third position, the region selector 52 illustratively connects the inlet 54 with each of the first, second, and third outlets 56a, 56b, 56c. As shown in FIG. 3B, the third outlet 56c is illustratively connected with a free section 60 of conduit 18 including a free end 62 having a connector 64 for selective connection with other parts of the ozone dispersion system 10 (disclosed in additional detail below) to communicate ozone thereto. Placing the region selector 52 in the third position illustratively connects each of the free section 60 and the conduit 18 of the upper and lower lower regions 33, 31 with the delivery system 16 to communicate ozone to other portions of the dispersion circuit, for example, portions connected with other garments, and to disperse ozone from each of the upper and lower regions 33, 31 and thus generally in the upper and lower regions of the jacket 12.

Accordingly, the region selector 52 permits selection of the regions of the ozone dispersion system 10 that receive communication of ozone. Such control promotes adaptability of the ozone dispersion to actual conditions and/or user preference. In a non-limiting example, the free section 60 can be connected with further conduit 18 of another garment, such as pants, to provide ozone cloaking of the pants. The region selector 52 permits selective adjustment to communicate ozone to pants that are fluidly connected with the free section 60 to receive and dispense ozone, as discussed in additional detail with regarding to FIGS. 23-34. Examples of such other garments and/or tools for fluid connection as a part of the dispersion circuit 14 may include hats, boots, blinds, gloves, and the like.

Returning to FIG. 1, the dispersion control system 48 is ergonomically positioned on the jacket 12 within comfortable reach of the user's arm. The flow control device 50 and region selector 52 are illustratively positioned near the user's right hip and/or abdomen. The dispersion control system 48 is illustratively shown rearward of a side pocket opening 35, but in some embodiments, may be arranged in any suitable manner, including relative to traditional garment features to promote access to multiple features. Ergonomic location of the dispersion control system 48 can provide ease of access. Such ease of access can reduce the potential for detection by reducing the amount and/or time of movement needed to adjust the control system 48, and/or reducing potential and/or degree of noise generated by adjustment of the control system 48.

As shown in FIG. 1, the conduit 18 illustratively includes sections 25a, 25b each extending from respective sections 22 towards the rear of the jacket 12 (about a user's right side beneath the arm of the jacket 12). Each section 25a, 25b illustratively connects with an additional section 23 disposed on the rear of the jacket 12 as shown in FIG. 5 to provide outlets 24 on the rear of the jacket 12 for dispersing ozone. The section 25a illustratively connects with section 23 on the rear of the jacket 12 arranged in the upper region 33 and the section 25b illustratively connects with section 23 on the rear of the jacket 12 that is arranged in the lower region 31 of the jacket 12. Ozone is illustratively provided through the sections 25a, 25b to their respective sections 23 on the rear of the jacket 12 according to the position of the region selector 52. In the illustrative embodiment, sections 29a, 29b extend respectively from the sections 25a, 25b toward the front of the jacket 12 (about the user's left side) to connect with sections 23 on the front left side (according to user's left side) of the jacket 12. In the illustrative embodiment, sections 23 in each area of the jacket 12 are housed within the dispersion guidance system 34 for assisting dispersion. In the illustrative embodiment, sections 23 within the same (upper and lower) region 33, 31 on the front right, front left, or rear of the jacket 12 are arranged at different distances from the face opening 32, but in some embodiments, may be equal distance from the face opening 32 for sections 23 in the same region 31, 33 and/or any other suitable arrangement to provide controlled ozone dispersion.

As shown in FIGS. 2 and 5, sections 23 on the rear of the jacket 12 are illustratively arranged within the space 38 of a respective dispersion guidance system 34, namely on the exterior 26 of the jacket 12 but covered by the respective flaps 36. The sections 22, 25a, 25b, 29a, 29b are illustratively housed within the interior of the jacket 21, at least within the exterior 26 of the jacket 12, and extend through the exterior 26 of the jacket 12 for connection with their respective sections 23. In some embodiments, the conduit 18 may be arranged in any suitable position relative to the jacket 12, for example but without limitations, interior and/or exterior to the jacket 12 in various areas.

Referring now to FIG. 4, the jacket 12 illustratively includes a pocket 70 which houses a power source 72 for providing electrical power to the delivery system 16. The pocket 70 is illustratively arranged on the left side of the jacket 12 (opposite the delivery system 16) with ergonomic positioning for access by the user. The power source 72 is illustratively embodied as a lithium ion battery, but in some embodiments, may include any suitable type of power source. The power source 72 illustratively includes a switch 74 positionable in either ON or OFF positions to activate or deactivate the power source 72. In the illustrative embodiment, the power source 72 includes a universal serial bus (USB) port 76 for connection of a USB cable 78 to electrically connect with the delivery system 16. As shown in FIG. 2, the USB cable 78 extends through the jacket 12 between the pocket 15 and the pocket 70 to electrically connect the power source 72 and the delivery system 16. The USB port 76 can also connect to a charging source to charge the power source 72, but in some embodiments, charging may be performed via another port. In some embodiments, the power source 72 may be connectable to a mobile device, such as a smart phone, through the USB port 76 or another USB port to provide power to charge the mobile device. In some embodiments, the power source 72 may be housed with the pocket 15 with delivery system 16 and/or be formed as part of the delivery system 16, for example, within or connectible with a common housing 80.

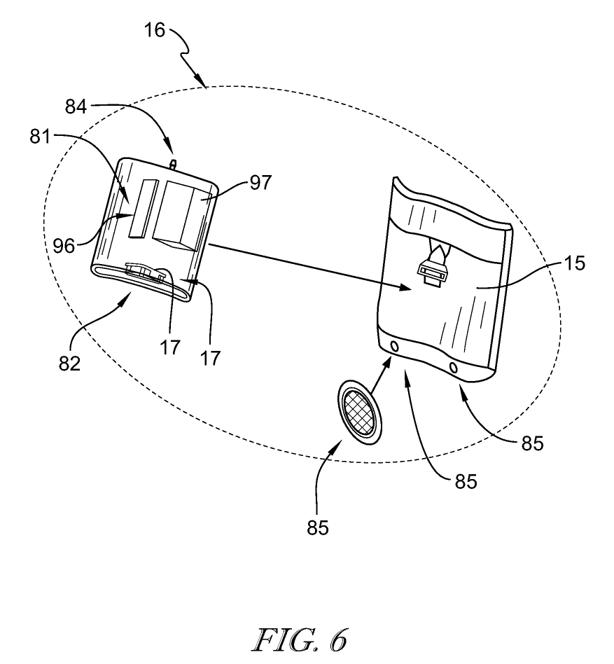

As mentioned above, the delivery system 16 is illustratively housed within the pocket 15 as suggested in FIG. 6. The delivery system 16 illustratively includes a housing 80 which contains components therein. The housing 80 is illustratively formed as a pressurizable container having a cavity 81 defined therein and an inlet 82 and an outlet 84 each fluidly connected with the cavity 81. The inlet 82 illustratively draws external air therethrough into the cavity 81 and the outlet 84 is connected with a portion of conduit 18 to pass pressurized fluid to the conduit 18. In the illustrative embodiment, the pocket 15 includes intake openings 85 formed therein to communicate external air with the inlet 82. The housing 80 can be held snugly in the pocket 15 by elastic straps within the pocket 15, by conforming shape of the pocket 15, and/or by any suitable manner to maintain stability within the pocket 15. Either or both of the inlet 82 and intake openings 85 may include screens or grids disposed therein to block ingress of foreign objects such as dirt.

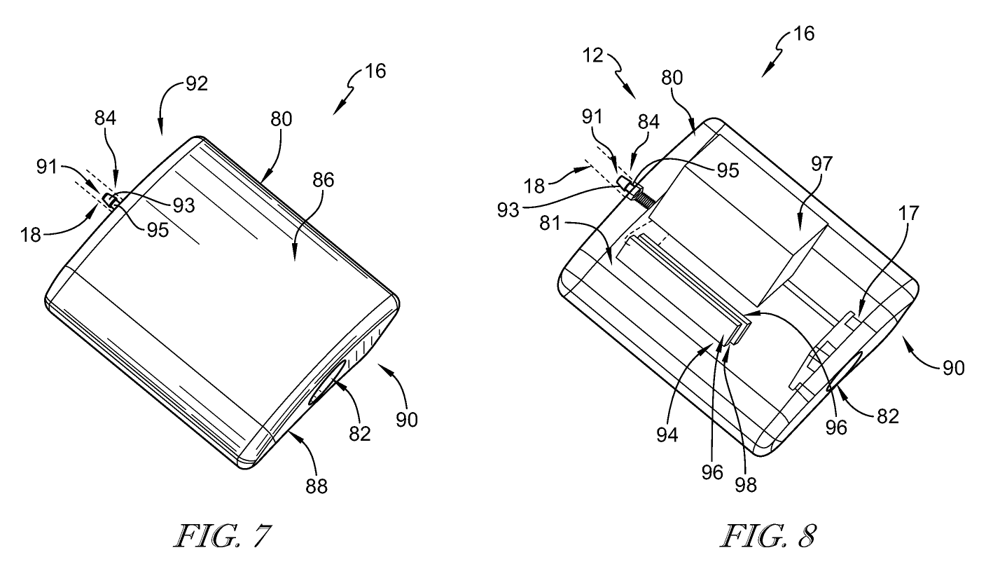

As shown in FIGS. 7-10, the housing 80 is illustratively formed to have curvature to promote comfort while being carried within the pocket 15. As shown in FIG. 7, a distal side 86 illustratively has a convex shape while an inner side 88 has a concave shape. The housing 80 illustratively has a flask shape but in some embodiments, may have any suitable shape. The inlet 82 is illustratively formed on one end 90 of the housing 80 and the outlet 84 is formed on another end 92, opposite the end 90.

In the illustrative embodiment as shown in FIGS. 7 and 8, the outlet 84 is formed as a male connector 91 having a through hole defined therein in fluid communication with the cavity 81. The male connector 91 is configured for connection with conduit 18 to communicate pressurized ozone. In the illustrative embodiment, the male connector 91 has a tapered head 93 with increasing diameter approaching the housing 80 and a neck 95 extending from the housing 80 to connect with the head 93 and having smaller diameter than the head 93 at their connection to form a step. The male connector 91 is illustratively configured for connection with the conduit 18 upstream of the flow control device 50, the conduit 18 forming a resilient female end receiving the male connector 91 therein for fluid connection of the delivery system 16 with the conduit 18. The conduit 18 female end may removably secure onto male connector 91 by resilient fit over the step between the head 93 and neck 95 of the male connector 91.

As shown in FIG. 8, the delivery system 16 illustratively includes the pressure source 17, embodied as an inlet fan, a generator module 94 for generating ozone as the cloaking substance, and power circuitry 97 for configuring power from the power source 72. The pressure source 17 draws air into the housing 80 to generate ozone (O₃) and to pressurize the cavity 81 to provide ozone to the dispersion circuit 14.

As shown in FIG. 8, the pressure source 17 is illustratively secured to the interior of the housing 80 on the end 90 at the inlet 82. The pressure source 17 illustratively draws external air through the inlet 82 into the cavity 81 as feed air for generating ozone and to pressurize the cavity 81 to distribute ozone. The generator module 94 illustratively generates ozone from at least a portion of the feed air.

In the illustrative embodiment, the generator module 94 is a corona discharge ozone generator, including a pair of electrodes 96 arranged to define a gap 98 therebetween. The electrodes 96 are embodied as plates arranged parallel to each other to define the gap 98 and charged to create a voltage differential to form a corona discharge across the gap 98. Feed air passing through the gap 98 is exposed to the corona discharge. Oxygen (O₂) within the feed air that is exposed to the corona discharge is converted to ozone (O₃). In the illustrative embodiment, the corona discharge includes a relatively high voltage-low current discharge, but in some embodiments, any suitable manner of ozone generation may be included, for example but without limitation, corona discharge and/or ultra violet light ozone generation. The generator module 94 thus creates ozone within the cavity 81 which is pressurized by the pressure source 17 and distributed through the outlet 84 to the dispersion circuit 14. In some embodiments, ozone may be available in advance and stored in a reservoir and provided to the conduit 18, for example, by concentrated feed into the cavity 81 for controlled distribution.

As shown in FIG. 8, the power circuitry 97 is illustratively connected with the generator module 94 to provide electric power to charge the electrodes 96. The power circuitry 97 illustratively connects with the USB cable 78 to receive power from the power source 72. The power circuitry 97 illustratively includes a transformer connected to increase the voltage and to configure the power received from the power source 72 for use in the generator module 94 to create the corona discharge. In some embodiments, the power circuitry 97 may include any number, type, and/or arrangement of components to provide suitable power to the generator module 94 for producing ozone. In the illustrative embodiment, the pressure source 17 uses power directly from the power source 72, but in some embodiments, the power circuitry 97 may include circuitry components arranged to configure power for operation of the pressure source 17.

In the illustrative embodiment, the pressure source 17, the power circuitry 97, and the generator module 94 are arranged securedly within the cavity 81. In some embodiments, any of the pressure source 17, the power circuitry 97, and the generator module 94 may be arranged outside of the housing 80 in communication with appropriate other components to provide pressurized ozone to the dispersion circuit 14. In some embodiments, the flow of feed air through the generator module 94 and to the outlet 84 may be piped for operational efficiency. In some embodiments, the rate of flow of feed air into the cavity (and thus the flow rate out from the cavity) may be adjustable by variable speed pressure source.

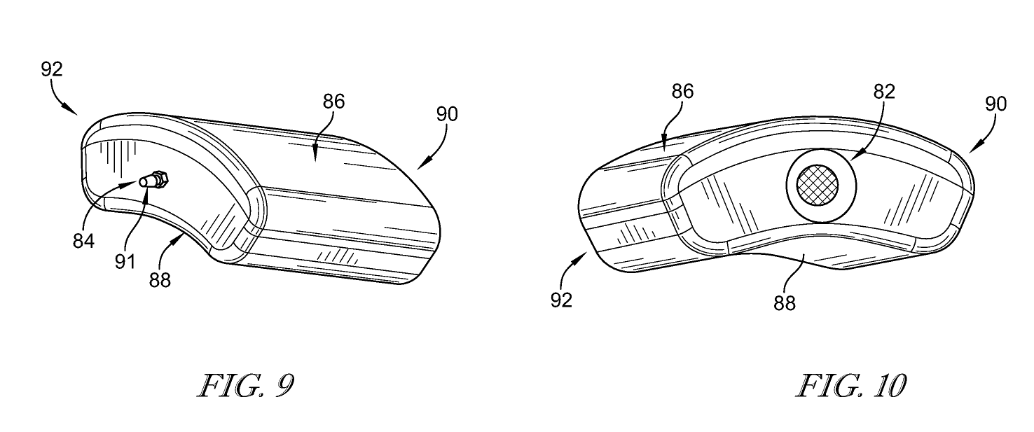

As shown in FIGS. 9 and 10, the flask shape of the housing 80 is apparent. The inlet 82 and outlet 84 are centrally located on their respectively ends 88, 90, but in some embodiments, may have any suitable arrangement to promote communication for communication of fluid. The outlet 84 is illustratively formed as an opening through the housing 80 having a tapered diameter to funnel air into the pressure source 17. The inlet 82 may include a covering, such as a screen, to block foreign substances from entering the cavity 81.

As shown in FIGS. 11-13, another illustrative embodiment of a delivery system 1016 for providing ozone to the dispersion circuit 14 is shown. The delivery system 1016 is illustratively similar to delivery system 16 and the disclosure of delivery system 16 applies to delivery system 1016, except where it would conflict with the specific disclosure of delivery system 1016. The delivery system 1016 illustratively includes a housing 1080, a pressure source 1017 formed as a pump, the generator module 94, and power circuitry 1095 for configuring power to the generator module 94. The housing 1080 illustratively includes an inlet 1082 and outlet 1084 (for connection with conduit 18) formed at opposite ends 1090, 1092. The housing 1080 is formed similar to housing 80 except that inlet 1082 can have a smaller diameter in communication with the pressure source 1017 formed as a pump.

As shown in FIG. 13, the housing 1080 defines a cavity 1081 connected with each of the inlet 1082 and outlet 1084. The pressure source 1017 illustratively draws external air into the housing 1080 for generation of ozone and to pressurize the cavity 1081. As mentioned above, the pressure source 1017 is embodied as a pump, namely a diaphragm pump, but in some embodiments, may be any suitable pressure device. The pressure source 1017 illustratively includes an intake 1045 connected with the inlet 1082 to receive the external air as feed air. The pressure source pressurizes the feed air and expels the feed air into the cavity 1081.

As shown in FIG. 13, the generator module 94 forms ozone from at least a portion of the feed air as described above. The power circuitry 1095 illustratively configures electric power from the power source 72 for use by the generator module 94. Ozone created by the generator module 94 is discharged under the pressure of the housing 1080 through the outlet 1084 to the conduit 18.

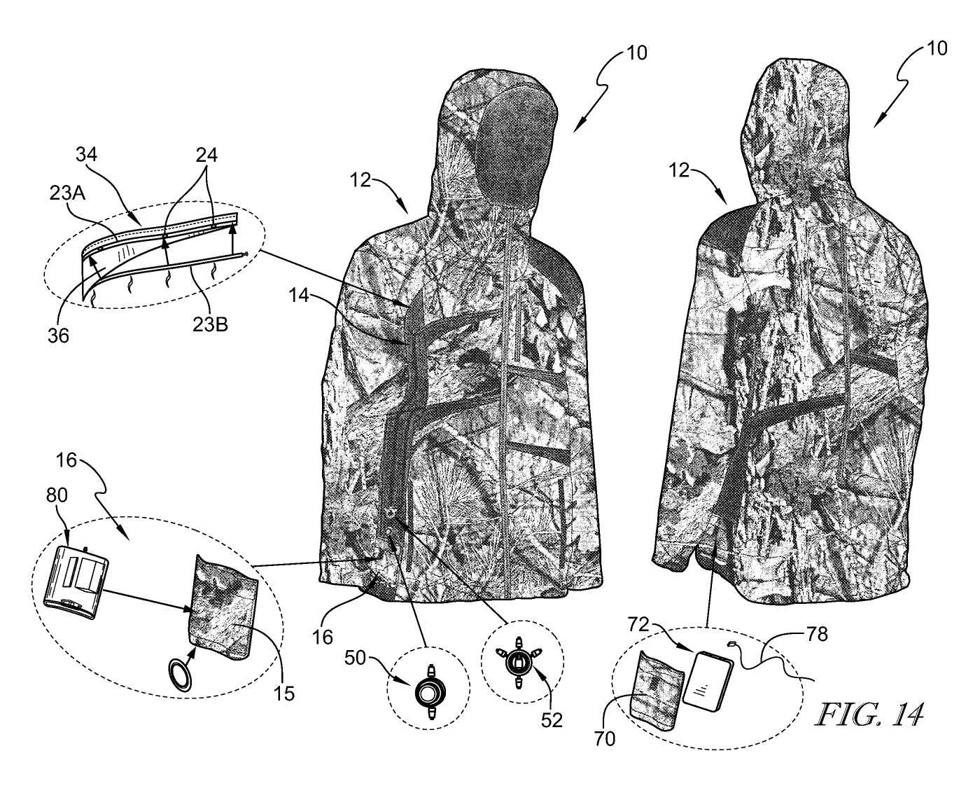

Referring to FIG. 14, the jacket 12 of FIG. 1 is shown having a camouflage pattern, illustratively a woodlands pattern, to inhibit detection by sight, although in some embodiments, any suitable pattern may be applied.

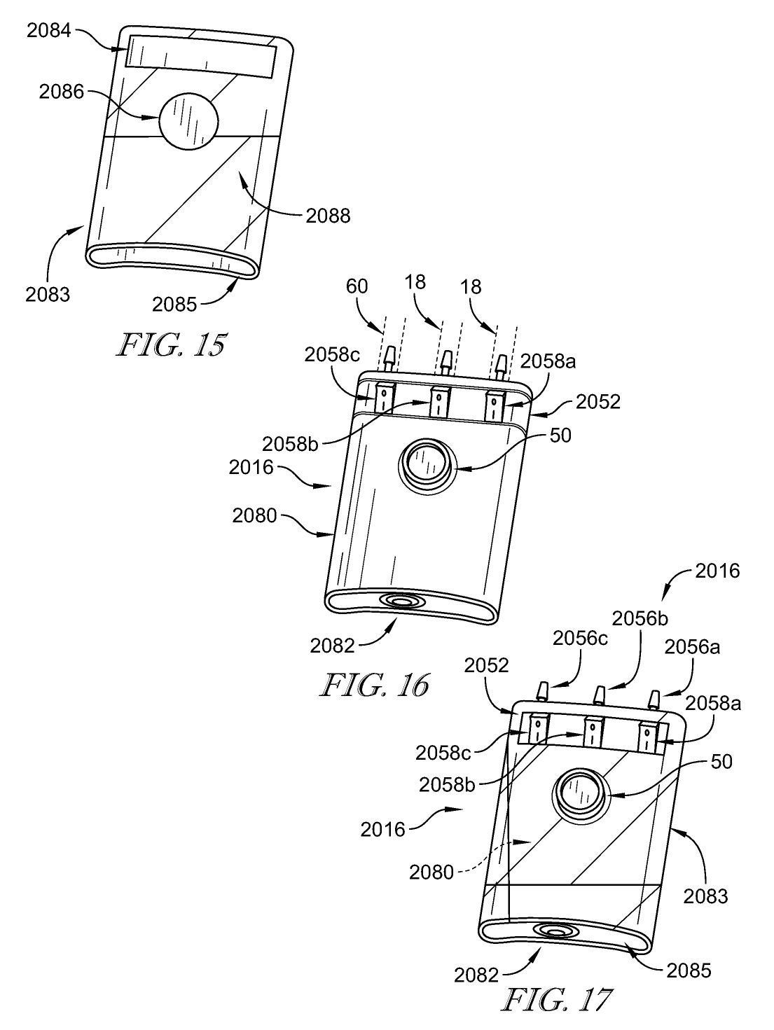

As shown in FIGS. 15-21, another illustrative embodiment of a cloaking substance delivery system 2016 for providing ozone to the dispersion circuit 14 is shown. The delivery system 2016 is illustratively similar to delivery systems 16, 1016 and the disclosure of delivery systems 16, 1016 applies to delivery system 2016, except where they would conflict with the specific disclosure of delivery system 2016.

As shown in FIG. 17, the delivery system 2016 illustratively includes a housing 2080 having a covering 2083 secured on an outside of the housing 2080. In the illustrative embodiment, the covering 2083 is formed of neoprene, but in some embodiments, may include any suitable materials. The covering 2083 illustratively includes access openings 2084, 2086, formed on a front side 2088 thereof as shown in FIGS. 15 and 17, for access to control devices. Referring to FIG. 16, the delivery system 2016 illustratively includes a dispersion control system 2048, including a flow control device 50 and region selector 2052 secured with the housing 2080 and extending outside the housing 2080 for adjustment access.

The delivery system 2016 illustratively includes outlets 2056a, 2056b, 2056c each connected to respective conduit sections to provide ozone to the lower region 31, upper region 33, and other garments respectively (as described above for outlets 56a, 56b, 56c of the region selector 52). The region selector 2052 illustratively includes flow switches 2058a, 2058b, 2058c each corresponding respectively to the outlets 2056a, 2056b, 2056c and each operable in either an ON or OFF position to fluidly connect or disconnect the housing 2080 with the respective outlet 2056a, 2056b, 2056c. The individual flows switches 2058a-c provide flexibility in selecting the region to which the ozone may flow in an integrated package with the housing 2080. The delivery system 2016 illustratively includes appropriate piping to distribute ozone generated therein through the flow control device 50 and the region selector 2052 to the dispersion circuit 14. In some embodiments, individual flow valves may control flow to each designated portion of the garment (e.g., by outlets 56a-c, 2056a-c), each valve being operable in variable positions from 0-100% of flow, without additional region selectors, with 0% flow positions permitting shutdown of ozone to a particular designated portion of the garment (or to other connected garments).

Referring to FIG. 17, the delivery system 2016 includes the pressure source 17, 1017 which illustratively draws external air as feed air through an inlet 2085 in the covering 2083 and the inlet 2082 in the housing 2080. The switches 2058a-c each extend through the access opening 2084 and the flow control device 50 extends through the access opening 2086 for access by the user outside of the covering 2083. The inlets 2082, 2085 are illustratively arranged to communicate with intake openings 85 of the jacket 12 to receive air from the atmosphere.

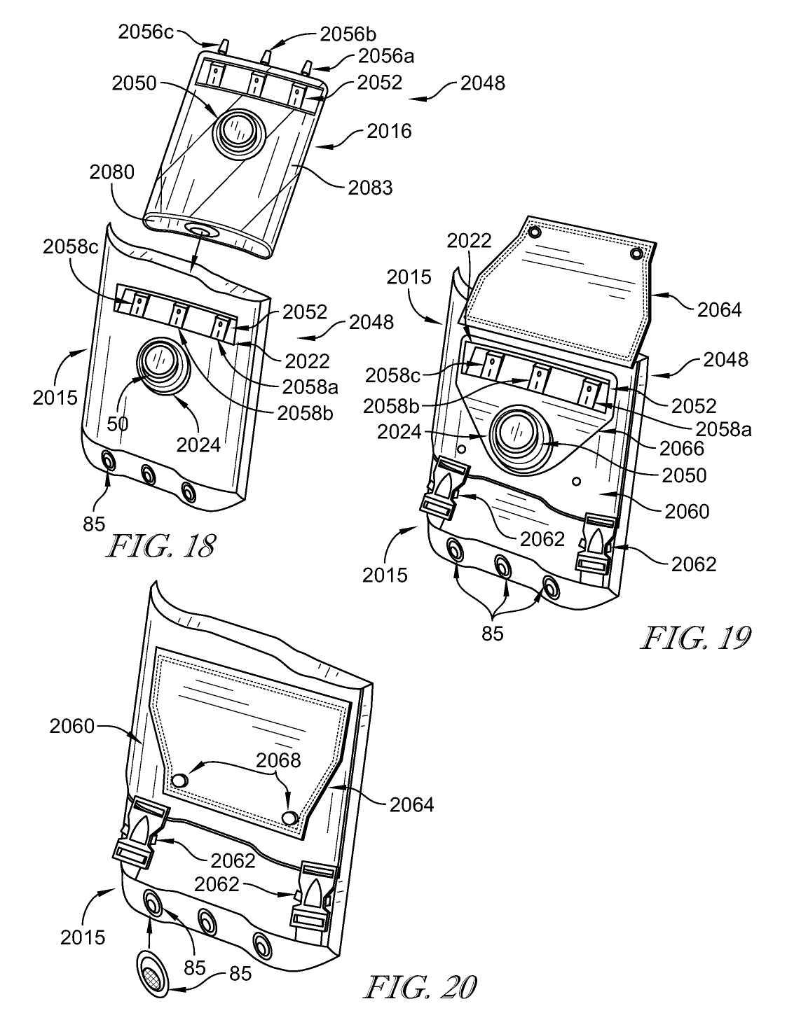

As shown in FIGS. 18-20, the delivery system 2016 is housed within a pocket 2015 as another embodiment, of pocket 15 of the jacket 12. FIG. 18 illustrates the delivery system 2016 being inserted into the pocket 2015 from the top such that when seated therein, the region selector 2052 is accessible through an opening 2022 and the flow control device 50 is accessible through an opening 2024 of the pocket 2015. As shown in FIG. 19, the housing 2080 is arranged within the pocket 2015 and an optional inner flap 2060 is secured into a closed position by buckles 2062. An outer flap 2064 is illustratively shown in an open position in FIG. 19 to show that the inner flap 2060 includes an operation opening 2066 to permit user operation of the dispersion control system 2048 through the operation opening 2066 while the inner flap 2060 is in the closed position to secure the housing 2080 within the pocket 2015.

As shown in FIG. 20, the outer flap 2064 is arranged in a closed position to cover the region selector 2052 and flow control device 50. The outer flap 2064 illustratively includes fasteners 2068, embodied as silent snaps, for securing the outer flap 2064 over the dispersion control system 2048 for easy release and movement into the open position for access to the dispersion control system 2048. In embodiments excluding the inner flap 2060 the outer flap 2064 can be secured to the pocket 2015 itself. Thus, a user can easily access the dispersion control system 2048 while providing protection, for example, from weather and/or inadvertent adjustment. In some embodiments, various coverings and/or fasteners may secure the housing 2080 while providing access to the dispersion control system 2048.

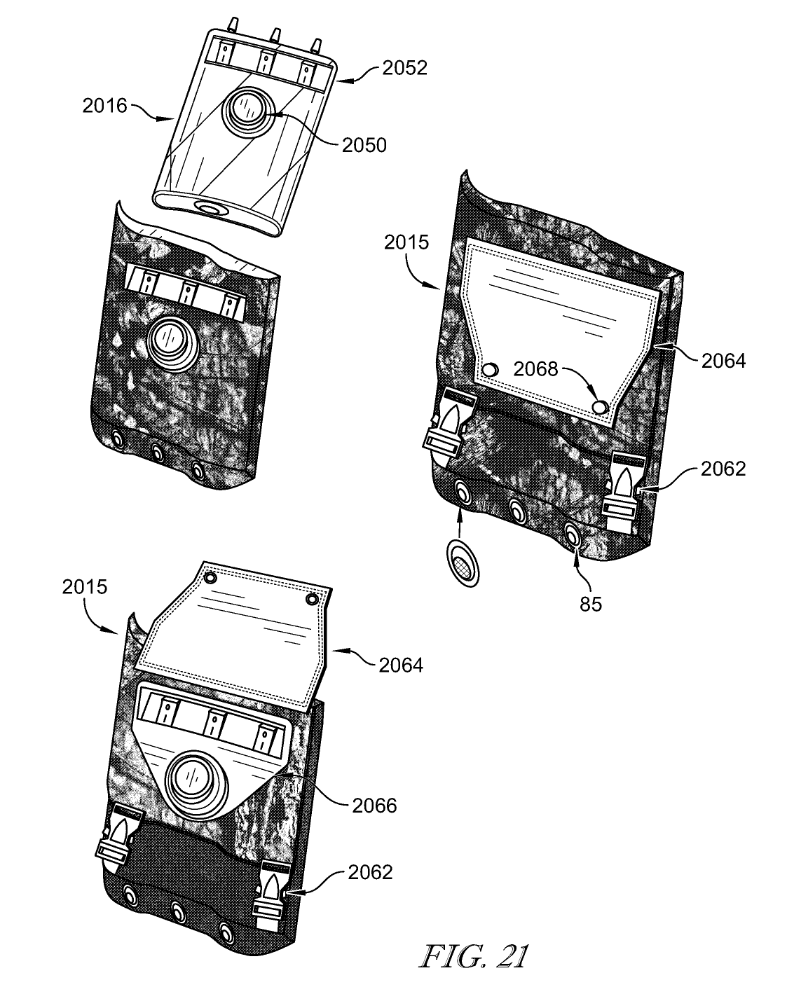

As shown in FIG. 21, the features of FIGS. 18-20 are shown with the jacket 12 generally rendered with camouflage, in a woodlands pattern, to inhibit sight detection, although in some embodiments, any suitable pattern may be applied.

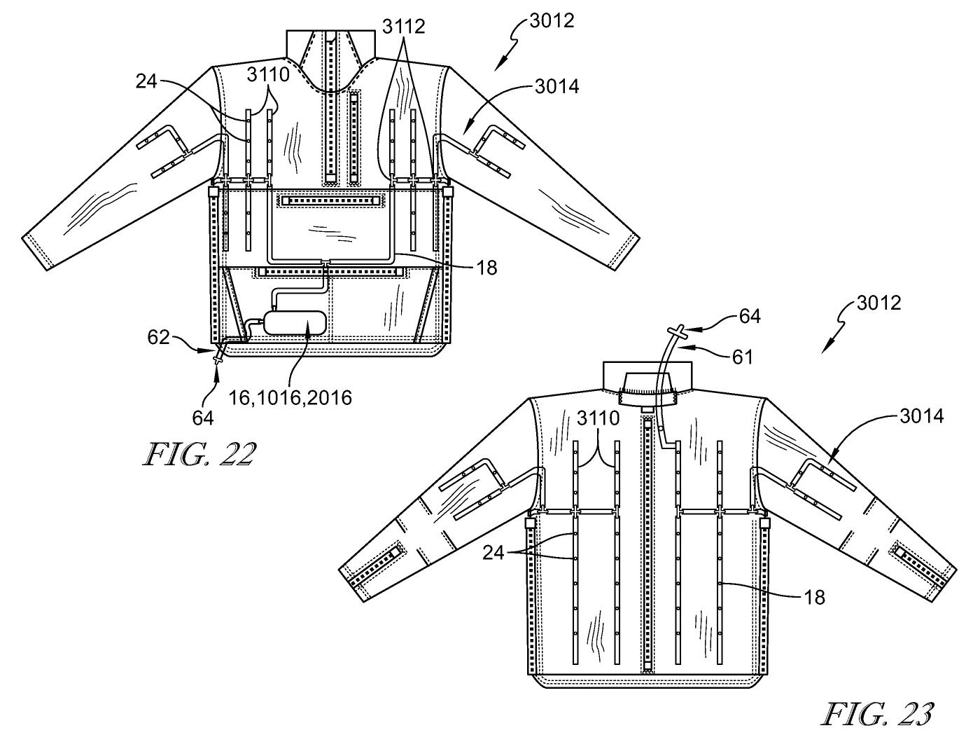

As shown in FIGS. 22 and 23, another illustrative embodiment of a garment is shown as jacket 3012. The jacket 3012 is similar to the jacket 12 and the disclosure of jacket 12 applies equally to the jacket 3012 except in instances of conflict with the specific disclosure of jacket 3012. The dispersion circuit 14 of jacket 3012 illustratively includes conduit 18 including perforated conduit pieces 3110 having outlets 24. The pieces 3110 are illustratively connected together by various splitters 3112 embodied as having male connector ends for insertion within the conduit pieces 3110. The dispersion circuit 14 connects with the cloaking substance delivery system 16, 1016, 2016 to receive cloaking substance for dispersion. Although a single connection between the delivery system 16, 1016, 2016 is shown without dispersion control system 48, 2048 in some embodiments, the control system 48, 2048 including flow control device 50 and/or region selector 52, 2052 may be included with connections to any number of suitable conduit portions. In the illustrative embodiment, the free end 62 is fluidly connected with the delivery system 16, 1016, 2016 and includes the connector 64 formed as a male connector, similar to male connector 91, for connection with other parts of the ozone dispersion system 10.

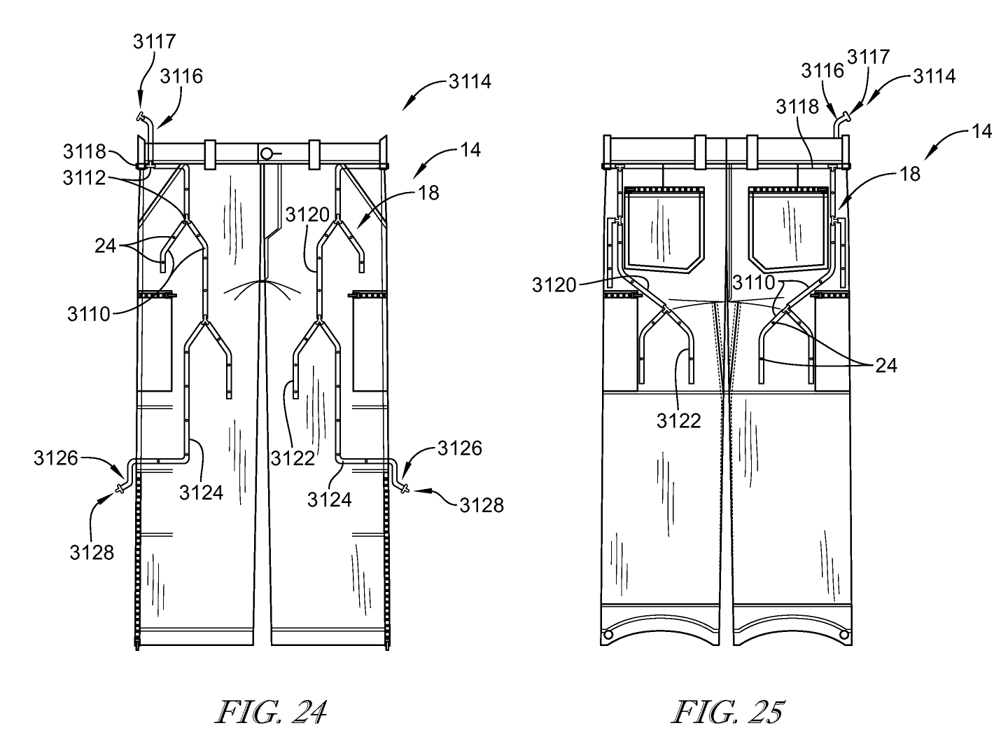

As shown in FIG. 24, a pair of pants 3114 is shown including conduit 18 for connection with the conduit 18 of the jacket 12, 3012 as a portion of the dispersion circuit 14. In the illustrative embodiment, the pants 3114 are a distinctively separate garment from the jacket 12, 3012, permitting the user to elect to wear only one or the other of the jacket 12, 3012 or pants 3114, and/or to more easily dress and undress from the garments. The free end 62 of the conduit 18 of the jacket 12, 3012 is selectively connectible (and separable) with a free end 3116 of the conduit 18 of the pair of pants 3114. The free end 3116 is illustratively embodied to have a female connector 3117 configured for complementary engagement with the male connector 64 to provide fluid communication. The pants 3114 can be employed together with the jacket 12, 3012 as a garment collection or system. In some embodiments, the jacket 12, 3012 and/or pants 3114 may have common fabric portions with each other or with other garments such as in the case of coveralls, garments with zipoff limbs, waders, garments with connected footwear (e.g., waders), and/or other less conventional and/or sporting specific garment types. Selective connection between the free ends 62, 3116 can provide fluid communication of cloaking substance from the delivery system 16, 1016, 2016 to portions of the dispersion circuit of the pants 3114.

The conduit 18 of the pants 3114 illustratively includes a number of conduit pieces 3110 having outlets 24 as shown in FIG. 24. The pieces 3110 are illustratively connected together by various splitters 3112 embodied as having male connector ends for insertion within the conduit pieces 3110. The free end 3116 is illustratively arranged near the user's right hip, complimentary in location to the dispersion delivery system 16, 1016, 2016 to reduce connection lengths and/or provide ergonomic access to the connections. The free end 3116 illustratively connects with section 3118 which extends around the user's side to connect with conduit 18 on the rear of the pants 3114 as shown in FIG. 25.

The conduit 18 of the pants 3114 is illustratively arranged in a vine style. The conduit 18 illustratively includes main pieces 3120 and branch pieces 3122 as shown in FIG. 24. The branch pieces 3122 illustratively terminate a channel of conduit 18, while the main pieces 3120 connect with one or more other pieces 3110. A main piece 3124 illustratively forms another free end 3126 for selective connection with further conduit of an additional garment, for example, footwear, to distribute ozone thereto. Each main piece 3124 includes a male connector 3128 arranged to connect with further conduit near the user's calf. Although the conduit 18 of the pants 3114 is shown secured on the exterior of the pants 3114, in some embodiments, one or more of the pieces 3110 may be arranged inside the pants 3114 allowing ozone to emanate through the pants 3114 and/or with one or more pieces 3110 on the exterior to directly emit ozone. Disclosed features of the jacket 12, 3012 and/or its related parts may be similarly applied to pants 3114 and/or other garments and/or tools discussed herein.

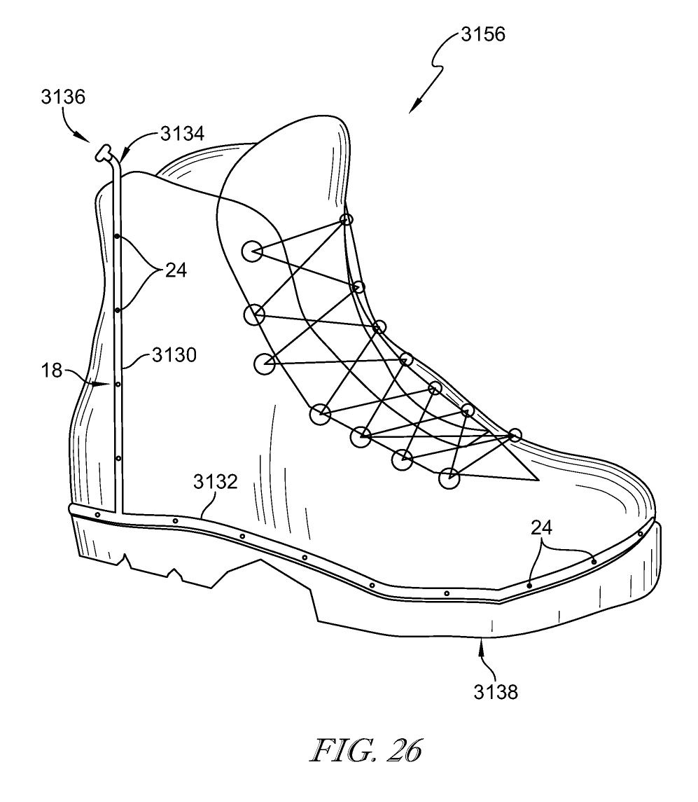

As shown in FIG. 26, the dispersion circuit 14, including the conduit 18 of the pants 3114, is selectively connectible with other garments, namely boots 3156. Each boot 3156 illustratively includes conduits 18 having sections 3130,3132 that are selectively connectible with other conduit 18 (e.g., on the pants 3114) to form a portion of the dispersion circuit 14 to communicate ozone. The conduits sections 3130,3132 on each boot 3156 illustratively include a free section 3130 having a free end 3134 with a female connector 3136. The connectors 3136 are each configured for selective connection with the respective connectors 3128 on the corresponding leg of the pants 3114 to provide fluid connection for communicating ozone through the sections 3130,3132 and the outlets 24 of each boot 3156 for cloaking each boot 3156 with ozone.

In the illustrative embodiment as shown in FIG. 26, the conduit of each boot 3156 illustratively include a section 3132 extending near the sole 3138 of the boot 3156 to arrange its outlets 24 in close proximity to the ground surface which the boot 3156 contacts. As with other portions of conduit 18, one or more of the sections 3130, 3132 may be internal to the boot to provide ozone permeating therethrough and/or with certain sections external to the boot. In some embodiments, the boots 3156 may be connected with the delivery system 16, 1016, 2016 by an auxiliary conduit 18 unattached to the pants 3114 to permit cloaking the boots 3156 while using pants unconnected and/or unequipped for communicating ozone.

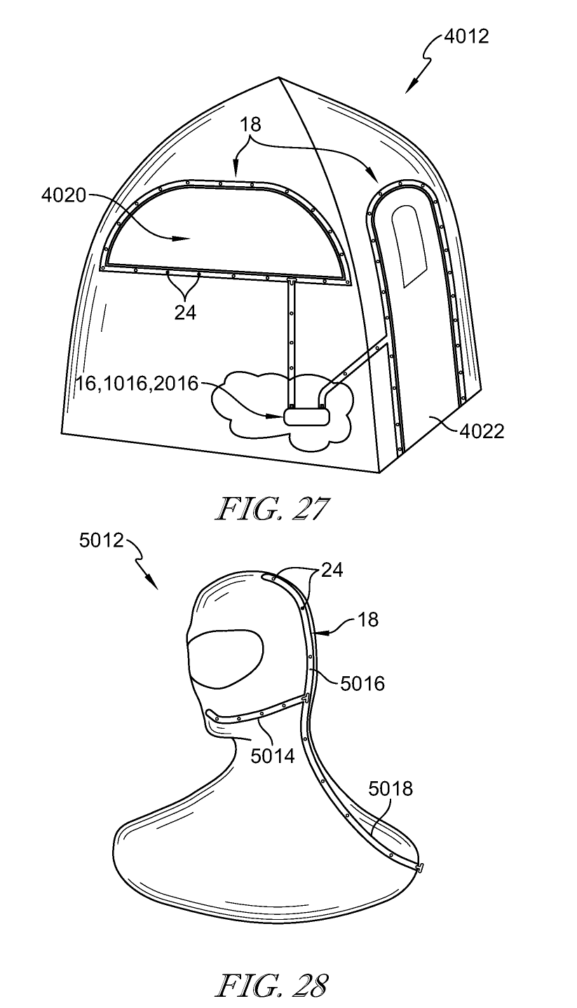

Another illustrative embodiment of an ozone dispersion system 10 is shown in FIG. 27 to include a tool, embodied as a hunting blind 4012, conduit 18 coupled with the blind 4012, and a cloaking substance delivery assembly 16, 1016, 2016 for providing ozone through the conduit 18 for emission to cloak the blind 4012 with ozone. In the illustrative embodiment, the conduit 18 includes window section 4016 and door section 4018 each disposed about a respective window 4020 and door 4022 of the blind 4012. The window and door sections 4016, 4018 are each illustratively connected with the delivery system 16, 1016, 2016 by a connection section 4024. Although the delivery system 16,1016, 2016 is illustratively shown isolated from the jacket 12, 3012 and other garments, in some embodiments, the delivery system 16,1016, 2016 may be connected simultaneously with any of the jacket 12, 3012, other garments, and blind 4012.

In the illustrative embodiment as shown in FIG. 28, a mask 5012 is shown including conduit 18 for connection with the conduit 18 of the jacket 12, 3012 as a portion of the dispersion circuit 14. The mask 5012 illustratively includes a mouth section 5014 extending along the user's cheek and a dome section 5016 extending towards the crown of the user's head. Each of the mouth and dome sections 5014,5016 are connected with a free end 5018 having a female connector for connection with a male connector 64 of a free end 61 of the jacket 12, 3012. The free end 61 of the jacket 12 is illustratively arranged inside the face opening 32 and connected with the conduit 18 of the upper section 33, as shown in FIG. 1, to permit selective distribution of ozone to the mask 5012 when connected to the dispersion circuit 14. The free end 61 may be selectively positionable through an opening to outside of the exterior 26 for connection as desired with other garments (e.g., backpack 6012).

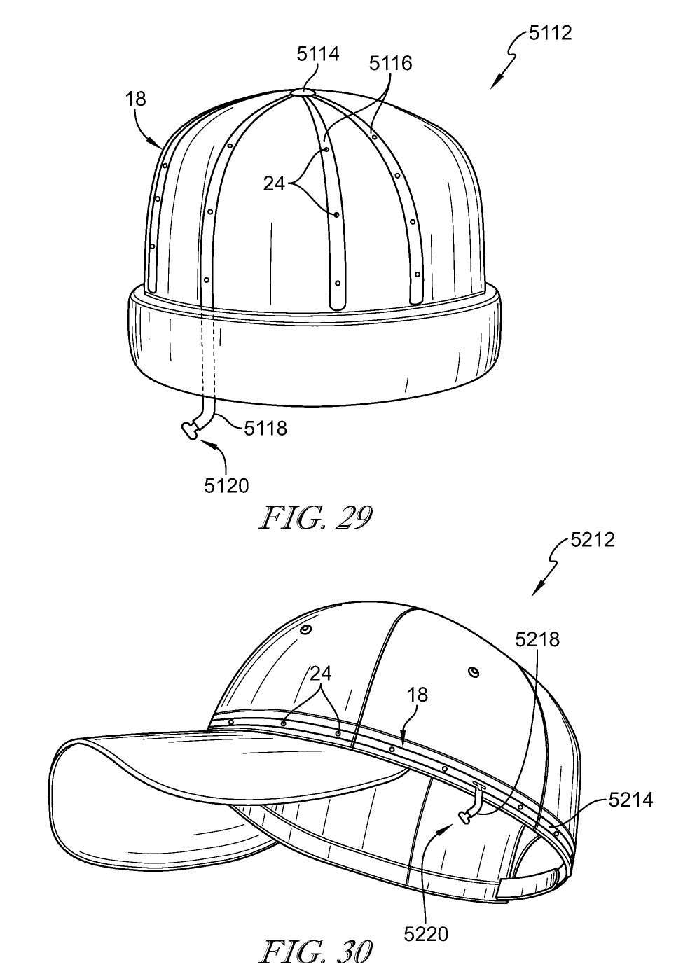

As shown in FIGS. 29 and 30, a beanie 5112 and a cap 5212 illustratively include conduit 18 for connection with the ozone delivery system 16, 1016, 2016. The beanie 5112 illustratively includes conduit hub 5114 arranged near the top of the user's head and connected with a number of finger sections 5116 extending from the hub 5114 to distribute ozone to the outlets 24 thereof. The cap 5212 illustratively includes a rim section 5214 having outlets 24 and extending about the user's head, The each of the beanie 5112 and the cap 5212 illustratively includes a free end 5118, 5218 having a female connector 5120, 5220 for connection with the connector 64 of the free end 61 of the jacket 12, 3012 to receive ozone from the ozone delivery system 16, 1016, 2016.

As shown in FIG. 31, another garment embodied as backpack 6012 is shown including compartments 6014, 6016 for storage and portage of items. The compartments 6014, 6016 are illustratively accessible through flap and/or zipper enclosed openings 6018, but in some embodiments, any suitable access manner may be applied. The backpack 6012 illustratively includes straps 6020 extending from an upper rear portion to a lower rear portion for hooking over the user's shoulders for carrying. The conduit 18 of the backpack 6012 includes having outlets 24 and comprises sections 6022, 6024, 6026 arranged respectively near each of the compartment openings 6018 and the straps 6020 to dispense ozone for cloaking the backpack 6012. Each section 6022, 6024, 6026 is illustratively connected with a free end 6028 having a female connector for connection with the conduit 18 of the jacket 12, 3012. In the illustrative embodiment, the free end 6028 is arranged near the user's neck and maybe connected with the free end 61 of the jacket 12, 3012.

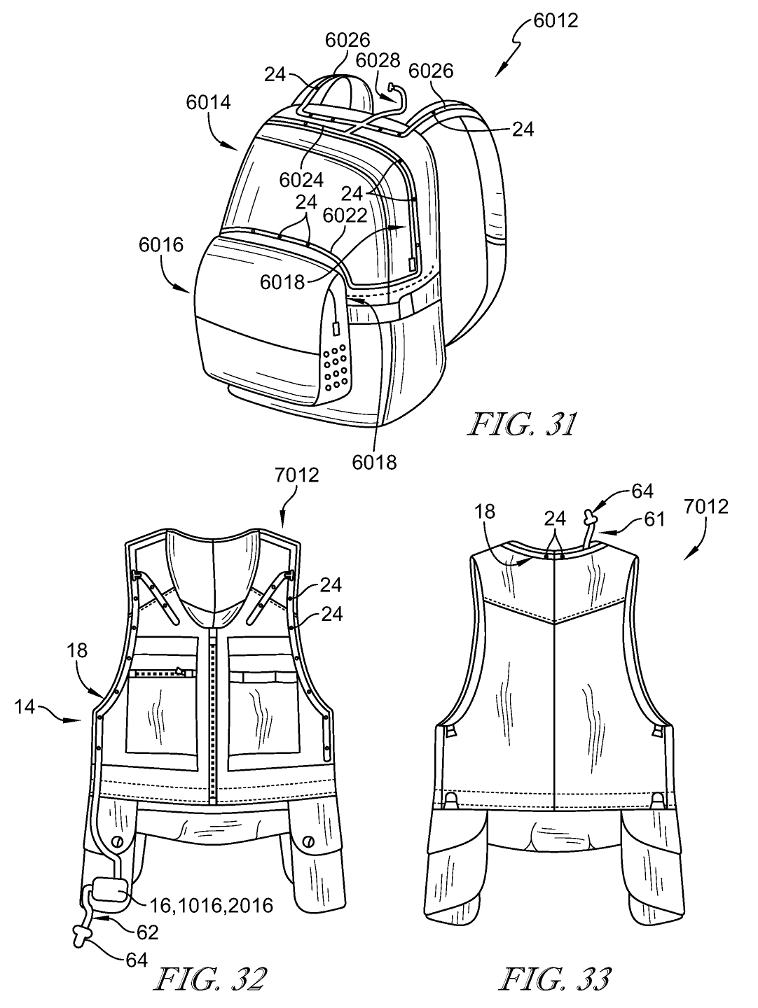

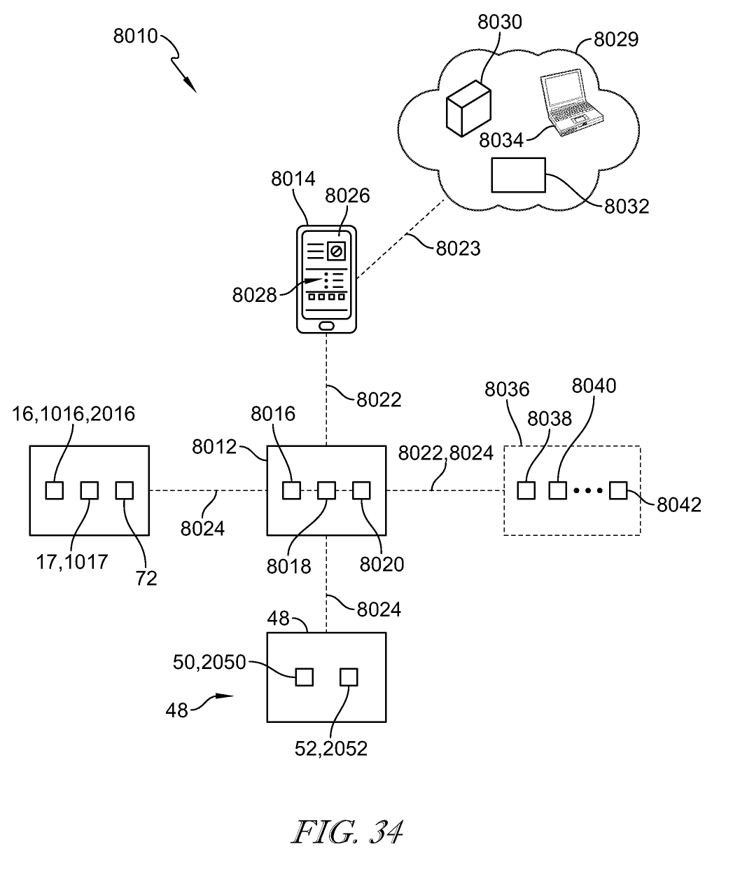

As shown in FIGS. 32 and 33, another garment, embodied as a hunting vest 7012, is shown including conduit 18 as a portion of the dispersion circuit 14 connected with the ozone delivery system 16, 1016, 2016 to provide ozone cloaking. The vest 7012 can be substituted for or used together with the jacket 12, 3012. The vest 7012 illustratively includes free ends 61,62 adapted for selective connection with other garments to provide ozone thereto.

In the illustrative embodiment, the various garments (jacket, vest, pants, footwear, hats, masks, etc.) are distinctive articles of clothing that are separate from each other, permitting the user to selectively wear only some of the articles, and/or to more easily dress and undress from the articles. Selective connection of conduit between various articles of clothing allows the user to selectively employ cloaking with various combination of garments without requiring any particular combination. In some embodiments, the cloaking substance delivery system 16, 1016, 2016 is selectively connectible directly with any particular garment herein to allow cloaking of that particular garment without one of the jacket 12, 3012 or vest 7012. Accordingly, an adaptable garment collection provides flexibility in dispersion of ozone and/or outfitting of the user according to desire and/or considitions.

In the illustrative embodiment as shown in FIG. 34, a communications system 8010 is shown providing communication with the dispersion control system 48, 2048 to govern ozone dispersion. The communications system 8010 can be applied with any of the disclosed dispersion systems, garments, tools, and there methods. The communications system 8010 illustratively includes a communications controller 8012 in communication with the dispersion control system 48, 2048 and a personal mobile device 8014. The communications controller 8012 can provide a communications hub to connect the personal mobile device with the dispersion control system 48 to provide the user with system information and/or control interfacing.

As shown in FIG. 34, the communications controller 8012 illustratively includes a processor 8016 for executing instructions stored on a memory device 8018. The communications controller 8012 includes communications circuitry 8020 adapted to communicate communication signals to and from the personal mobile device 8014 and the dispersion control system 48, 2048. In the illustrative embodiment, the communications controller 8012 includes component hardware (e.g., antenna, transceiver, etc.) and/or software (e.g., software, firmware, ect.) to establish wireless communication via link 8022 with the personal mobile device 8014.

The personal mobile device 8014 is embodied as a smart phone, but in some embodiments, may include any personal electronic device, including but not limited to personal digital assistants, wearables (e.g., glasses, watches, etc.), tablet and/or laptop computers, or the like. The link 8022 is illustratively embodied as a Bluetooth Low Energy (BLE) link. In some embodiments, the link 8022 may include any suitable type of wireless link, for example but without limitation, other Bluetooth versions, infrared, Wi-Fi, WiMax, Ultra Wideband, RFID, Zigbee, cellular (2G,3G,4G,5G), and/or similar, analogous, and/or comparable wireless technologies. In some embodiments, the link 8022 may include an optional hardwire connection with the controller 8012. The communications controller 8012 is illustratively connected with the dispersion control system 48, 2048 via link 8024. The link 8024 is embodied as a hardwire link, but in some embodiments, may include any suitable wireless link.

The user can illustratively view and adjust parameters of the dispersion control system 48, 2048 on the personal mobile device, for example, the positions (0-100%) of the flow control device 50, 2050 and/or region selector 52, 2052 are communicated to the personal mobile device 8014. The parameters are illustratively displayed on a display screen 8026 of the mobile device 8014, embodied as a touch screen. Adjustment controls 8028 can be displayed on the screen 8026 for receiving user input to adjust the position (0-100%) of the flow control device 50, 2050 and/or region selector 52 as ozone parameter settings. In some embodiments, the adjustment controls 8028 may provide indirect adjustment of the parameters, for example, by receiving inputs for determination of preferred ozone parameter settings by the communications controller 8012. The controller 8012 may communicate command signals to the dispersion control system 48, 2048 to achieve the preferred ozone parameter settings. For example, a user may input a preferred ozone concentration range (low, medium, high) and/or other preferences and/or input factors for determination of a preferred ozone cloaking parameters (e.g, hunting position height above the ground, landscape information (tree/plant density surrounding the user, weather conditions, geographic location, time/date, etc.) game type, equipment characteristics (type: (e.g., bow (compound, longbow, cross-bow), firearm (e.g., bolt, semi-auto, pump), ammunition, range), etc.), among others. Certain input factors may be obtained by the personal mobile device directly, for example, by onboard components, such as gps position and/or speed tracking, and/or from other sources discussed below.

In the illustrative embodiment as shown in FIG. 34, the personal mobile device 8014 can be arranged in communication with at least one network 8029. The network is embodied as a wide area network, such as the Internet, and may include connection with any of servers 8030, databases 8032, and/or terminals 8034. The personal mobile device 8014 can illustratively communicate information with the network 8029 via wireless link 8023 which is embodied as a cellular connection, but may include any suitable wireless link. For example but without limitation, the personal mobile device may communicate user inputs (e.g., equipment characteristics: compound bow) to the network 8029 and may receive information (e.g., compound bow effective range, weather information) from the network 8029. The personal mobile device 8014 can communicate information to the communications controller 8012 for determining preferred ozone parameter settings.

The communications controller 8012 can communicate with various optional (relatively) local information sources 8036. Local information sources 8036 can include wind sensors (e.g., wind speed, direction, temperature, humidity sensors) 8038, cloaking substance sensors 8040 and/or game trackers (e.g., cameras, thermal cameras) 8042, among other local information sources. Such local information sources may include devices deployed by the user in relatively local proximity to the hunting area to obtain particularly local information, for example, trail cameras deployed near the target hunting area. Local information sources 8036 may communicate with communications controller 8012 by wireless and/or wired links 8022, 8024.

The cloaking substance sensors 8040 may include an ozone sensor 8040 adapted to detect an amount of ozone. Ozone sensors 8040 can be deployed around the hunting area and/or on the user's person for ozone level monitoring. For example but without limitation, an ozone sensor 8040 may be arranged near the face opening 32 of the jacket 12, 3012 to determine the concentration of ozone near the user's face. The ozone sensor 8040 can communicate with the communications controller to determine whether a threshold level of ozone is present near the user's face. The communications controller 8012 can receive an indication of ozone level from the ozone sensor 8040 and may determine a detected level of ozone. If the detected level of ozone is equal to or greater than a threshold level of ozone, the communications controller 8012 can provide an alert to the user, for example, by an audio, haptic, and/or visual alert provided on the personal mobile device 8014. In some embodiments, the threshold level of ozone can include a predetermined concentration and/or concentration detected for a predetermined period of time (e.g., equal to or greater than a threshold for at least 90 seconds, and/or more than 3 instances within 15 minute period, etc.). In some embodiments, multiple thresholds levels may be applied simultaneously. In some embodiments, a distinct ozone level determination device may be applied in communication with any of the ozone sensor 8040 and personal mobile device 8014 for determining the sensed ozone level.

As shown FIG. 34, the communications controller 8012 is illustratively arranged in communication with either of the ozone delivery system 16, 1016, 2016 and/or power source 72. The communications controller 8012 can illustratively provide status information of the ozone delivery system 16, 1016, 2016, pressure source 17, and/or power source 72 for display on the personal mobile device 8014. The communications controller 8012 may receive power from the power source 72 and/or may have separate power source. Status information can include remaining charge level (%) of the power source 72, operational status of the ozone delivery system 16, 1016, 2016 (e.g., on/off, efficiency, mode (low, medium, high), etc.). In the illustrative embodiment, the communications controller 8012 is connected with the ozone delivery system 16, 1016, 2016 and/or power source 72 by wired link 8024, but in some embodiments, may include wireless link.

As mentioned above, the communications controller 8012 can determine preferred ozone parameter settings, for example, preferred positions of the ozone flow control device 50, 2050 and/or of the region selector 52, 2052, and/or preferred on/off operation and/or or preferred variable speed operation of the pressure source 17,1017, for example, low, medium, or high setting of fan/pump speed according to preferred parameters. The controller 8012 can communicate command signals to the dispersion control system 48, 2048 and/or pressure source 17, 1017 to achieve the preferred ozone parameter settings. For example, in embodiments including electrically actuated, automatically controlled devices 50, 2050, 52, 2052, the controller 8012 communicates command signals to the respective devices 50, 2050, 52, 2052 to obtain their respective positions to achieve the preferred ozone parameter settings. The controller 8012 may continuously or periodically update the preferred ozone parameter settings based on available information. In embodiments including local information sources 8036, such as ozone sensors 8040, the controller 8012 may determine adjust the respective positions of the devices 50, 2050, 52, 2052 continuously or periodically based on the local information sources 8036. For example, the communications controller 8012 may determine an optimal range of ozone concentration and operate the dispersion control system 48, 2048 and/or pressure source 17, 1017 to maintain ozone conditions within that range based on information from the ozone sensors 8040.

The present disclosure includes devices, systems, and methods of scent management including dispersion of cloaking substances to inhibit scent detection. The present disclosure includes scent management including dispersion of cloaking substances to decrease detection, attract game animals, repel insects, and/or otherwise promote the hunting experience, for example but without limitation, dispersion of game hormones, game scents, insect repellents, food and/or nutrient sources, other attractants, other repellents, and/or precursors and/or indicators of the same, and/or combinations thereof. In the illustrative embodiment, ozone is created by the generator module 94 using corona discharge, but in some embodiments, the ozone dispersion system 10 may include configuration for ozone generation by any suitable manner, for example but without limitation, ultraviolet radiation, electrolysis, radiochemical, and/or combinations thereof. In some embodiments, pre-formed ozone may be provided from a reservoir to the conduit 18.

In some embodiments, any of the garments and/or tools of the present disclosure may include individual flow control devices and/or selectors to provide garment/tools specific flow governance, alone or in combination with those of the dispersion control system 48, 2048. Free ends of the conduit of the present disclosure may include end caps for isolating and/or protecting open connectors and conduit ends during periods of non-use, for example, when remaining unconnected with other free ends.

In some embodiments, all male connectors may have similar shape and size, and all female connectors may have similar shape and size such that each male connector can be inserted into each female connector, but in some embodiments, some male connectors may be shaped and size complimentary with only specific female connectors to limited the possible connections. In some embodiments, female connectors may comprise resilient tube ends for stretch fitting over the head of male connectors. In some embodiments, female connectors may comprise rigid components, complimentarily shaped to correspond with respective male connectors to for fluid tight fit. In some embodiments, conduit of various garments and/or tools may be fluidly connected by any suitable manner.

Hardware and/or software components necessary to, complimentary to, applicable to, comparable to, and/or otherwise operable to achieve the form and function of the systems, devices, and methods herein, for example, are within the scope of the present disclosure.

Conduit 18 within the present disclosure is illustratively secured with the respective garment. Conduit portions which are arranged on the exterior of the garment are illustratively sewn into place, but in some embodiments, may be secured by any of snaps, straps, fasteners, adhesive, heat bond, and/or any other suitable joining manner. In the illustrative embodiment, the conduit 18 is secured with the respective garment along its extension length to remain relatively fixed in place relative to the garment. In some embodiments, portions of the conduit 18 of any of the garments may be replacably attached without destruction of its joining manner.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

What is claimed is:

1. A garment system for dispersion of cloaking substance comprising
at least one garment for wearing by a user, the at least one garment including a jacket,
a dispersion circuit coupled with the jacket, the dispersion circuit defining a dispersion pathway including at least one inlet and at least one outlet,
a dispersion guidance system secured with the jacket proximate the at least one outlet to direct flow of cloaking substance away from a user's face, and
a cloaking substance delivery assembly fluidly connected with the at least one inlet of the dispersion circuit to conduct cloaking substance through the dispersion pathway for discharge through the at least one outlet to cloak the jacket with cloaking substance.

2. The garment system of claim 1, wherein the at least one garment includes at least one other garment, each of the jacket and the at least one other garment having conduit, the conduit of the jacket being selectively connected with the conduit of the at least one other garment to define the dispersion circuit for passing cloaking substance to cloak the at least one garment.

3. The garment system of claim 1, wherein the dispersion guidance system includes a flap extending over an exterior of the jacket to define a pocket space, wherein the at least one outlet is arranged within the pocket space.

4. The garment system of claim 3, wherein the dispersion guidance system includes an opening formed between the flap and the exterior of the jacket for guiding cloaking substance out from the outlet.

5. The garment system of claim 4, wherein the opening is directed away from a face opening of the jacket to direct the cloaking substance away from the user's face.

6. The garment system of claim 4, wherein the opening is directed downward.

7. The garment system of claim 1, further comprising a dispersion control system including a region selection device connected with the dispersion circuit and operable for selective communication with at least two regions of the garment to selectively provide cloaking substance to the at least two regions.

8. The garment system of claim 7, wherein the region selection device is operable in a first position to communicate cloaking substance with the at least two regions and in a second position to block communication of cloaking substance to at least one of the at least one regions.

9. The garment system of claim 7, wherein one of the at least two regions is an upper region of the jacket and another of the at least two regions is lower region of the jacket.

10. The garment system of claim 7, wherein one of the at least two regions is a region of another garment having conduit that is selectively connected as part of the dispersion circuit and the region selection device is connected with the dispersion circuit to selectively provide cloaking substance to the region of the another garment.

11. A garment ozone dispersion system for providing a cloak of ozone to prevent detection by game, the garment ozone dispersion system comprising
at least one garment for wearing by a user,
a dispersion circuit coupled with the at least one garment, the dispersion circuit defining a dispersion pathway including at least one inlet and at least one outlet,
a dispersion guidance system secured with the at least one garment proximate the at least one outlet to direct flow of cloaking substance away from a user's face, and
a cloaking substance delivery assembly fluidly connected with the at least one inlet of the dispersion circuit to conduct cloaking substance through the dispersion pathway for discharge through the at least one outlet to the dispersion guidance system to cloak the garment with cloaking substance.

12. The garment ozone dispersion system of claim 11, wherein the at least one garment includes a plurality of garments each having conduit, the conduit of each one of the plurality of garments being selectively connected with the conduit of the remainder of the plurality of garments to define the dispersion circuit for passing cloaking substance to cloak the plurality of garments.

13. The garment ozone dispersion system of claim 11, wherein the dispersion guidance system includes a flap extending over an exterior of the at least one garment to define a pocket space, wherein the at least one outlet is arranged within the pocket space.

14. The garment ozone dispersion system of claim 13, wherein the dispersion guidance system includes an opening formed between the flap and the exterior of the at least one garment for guiding cloaking substance out from the outlet.

15. The garment ozone dispersion system of claim 14, wherein the opening is directed away from a face opening of the garment to direct the cloaking substance away from the user's face.

16. The garment ozone dispersion system of claim 14, wherein the opening is directed downward.

17. The garment ozone dispersion system of claim 11, further comprising a dispersion control system including a region selection device connected with the dispersion circuit and operable for selective communication with at least two regions of the garment to selectively provide cloaking substance to the at least two regions.

18. The garment ozone dispersion system of claim 17, wherein the region selection device is operable in a first position to communicate cloaking substance with the at least two regions and in a second position to block communication of cloaking substance to at least one of the at least one regions.

19. The garment ozone dispersion system of claim 17, wherein one of the at least two regions is an upper region of the garment and another of the at least two regions is lower region of the garment.

20. The garment ozone dispersion system of claim 17, wherein one of the at least two regions is a region of another garment having conduit that is selectively connected as part of the dispersion circuit and the region selection device is connected with the dispersion circuit to selectively provide cloaking substance to the region of the another garment.
